(19) United States
(12) Patent Application Publication
(10) Pub. No.: US 2026/0215804 A1
(43) Pub. Date: Jul. 30, 2026

(54) STEERABLE LEAFLET RESECTION DEVICE

(71) Applicant: Medtronic, Inc., Minneapolis, MN (US)

(72) Inventor: Grant MENON, Cupertino, CA (US)

(21) Appl. No.: 19/147,365

(22) PCT Filed: Jan. 19, 2024
(86) PCT No.: PCT/IB2024/050530

§ 371 Date: Jul. 11, 2025

Related U.S. Application Data
(60) Provisional application No. 63/481,840, filed on Jan. 27, 2023.

(51) Int. Cl. A61B 17/3207; A61B 17/00

(52) U.S. Cl. CPC A61B 17/3207; A61B 17/00234; A61B 2017/00243; A61B 2017/00323

(57) ABSTRACT

A steerable leaflet resection device includes a first jaw (110) and a second jaw (130) coupled to the first jaw such that the first and second jaws include a closed configuration and an open configuration relative to each other. The steerable leaflet resection device further includes a first cutter (118) and a second cutter (138), each configured to slide relative to the first and second jaws. The first and second jaws are steerable into a first position wherein the first and second jaws are bent such that sliding the first cutter relative to the first and second jaws cuts the leaflet in a first curved path and are steerable into a second position different than the first position wherein the first and second jaws are bent such that sliding the second cutter relative to the first and second jaws cuts the leaflet in a second curved path different than the first curved path.

### CROSS-REFERENCE TO RELATED APPLICATION

This application claims the benefit of U.S. Provisional Patent Application Ser. No. 63/481,840, filed Jan. 27, 2023, the entire content of which is incorporated herein by reference.

### FIELD OF THE INVENTION

The present invention relates to a steerable leaflet resection device.

### BACKGROUND

Deployment of a second transcatheter heart valve prosthesis within a previously implanted surgical or transcatheter heart valve prosthesis is referred to as a valve-in-valve procedure. At a native aortic valve, coronary artery obstruction may occur when leaflets of the first implanted valve (or the native valve) are displaced towards the coronary ostia or sinotubular junction during deployment of the second transcatheter valve deployment, thus obstructing coronary blood flow. Thus, there is a need of a device and method to remove/resect the leaflets of the previously implanted heart valve prosthesis or the native valve leaflets.

### BRIEF SUMMARY OF THE INVENTION

In accordance with a first example hereof, a steerable leaflet resection device includes a first jaw disposed at a distal end of the leaflet resection device and a second jaw coupled to the first jaw. The first jaw and the second jaw are configured to move relative to each other into a closed configuration wherein distal end of the first and second jaws are disposed adjacent to each other such as to secure a leaflet of a valve therebetween and an open configuration wherein the distal end of the first and second jaws are spaced apart from each other such as to enable the first and second jaws to move relative to the leaflet with the leaflet disposed therebetween. The steerable leaflet resection device further comprises a first cutter configured to slide relative to the first and second jaws and a second cutter configured to slide relative to the first and second jaws. The first and second jaws are steerable into a first position wherein the first and second jaws are bent such that sliding the first cutter relative to the first and second jaws cuts the leaflet in a first curved path. The first and second jaws are then steerable into a second position different than the first position wherein the first and second jaws are bent such that sliding the second cutter relative to the first and second jaws cuts the leaflet in a second curved path different than the first curved path.

In a second example, in the steerable leaflet resection device of the first example, the first cutter is coupled to the first jaw and the second cutter is coupled to the second jaw.

In a third example, in the steerable leaflet resection device of the first or second examples, at least one of the first jaw and the second jaw comprising a hollow, discontinuous tube forming a longitudinal groove facing the other one of the first jaw and the second jaw, wherein at least one of the first cutter and the second cutter is slidably disposed within the tube such that least one of a first blade of the first cutter and a second blade of the second cutter is disposed through the longitudinal groove towards the other one of the first jaw and the second jaw.

In a fourth example, in the steerable leaflet resection device of any of the first through third examples, the steerable leaflet resection device further includes an anchoring tip disposed between the first jaw and the second jaw, wherein the anchoring tip is configured to secure the leaflet when the first and second jaws are in the closed configuration.

In a fifth example, in the steerable leaflet resection device of the fourth example, the anchoring tip extends from a distal end of the first jaw towards a distal end of the second jaw or from the distal end of the second jaw towards the distal end of the first jaw.

In a sixth example, in the steerable leaflet resection device of any of the preceding examples, the steerable leaflet resection device further includes at least a first steering wire and a second steering wire configured to bend the first and second jaws into the first position and the second position.

In a seventh example, in the steerable leaflet resection device of any of the preceding examples, the steerable leaflet resection device further includes a first steering wire coupled to a first side of the first jaw, a second steering wire coupled to a second side of the first jaw, a third steering wire coupled to a first side of the second jaw, and a fourth steering wire coupled to a second side of the second jaw, wherein tensioning the first and third steering wires bends the first and second jaws into the first position and tensioning the second and fourth steering wire bends the first and second jaws into the second position.

In an eighth example, in the steerable leaflet resection device of any of the preceding examples, the first jaw comprises a first hollow, discontinuous tube forming a first cavity and a first longitudinal groove facing the second jaw, the second jaw comprising a second hollow, discontinuous tube forming a second cavity and a second longitudinal groove facing the first jaw, the first cutter comprising a first base slidably disposed within the first cavity and a first blade extending through the first longitudinal groove towards the second jaw, and the second cutter comprising a second base slidably disposed within the second cavity and a second blade extending through the second longitudinal groove towards the first jaw.

In a ninth example, in the steerable leaflet resection device of the eighth example, the first blade and the second blade comprises sharp tips configured to puncture a leaflet.

In a tenth example, in the steerable leaflet resection device of the eighth example, the first blade and the second blade comprise sharpened edges comprised to cut through a leaflet as the first blade or the second blade is moved relative to the leaflet with the leaflet captured between the first and second jaws.

In an eleventh example, a method of resection a portion of a leaflet of a valve of interest includes tracking a steerable leaflet resection device through a vasculature of a patient to a valve of interest, wherein the steerable leaflet resection device includes a first jaw, a second jaw, a first cutter, and a second cutter; positioning the steerable leaflet resection device adjacent a free edge of a leaflet of the valve of interest; advancing the steerable leaflet resection device with the first and second jaws in an open configuration over the leaflet such that the leaflet is disposed between the first and second jaws; closing the first and second jaws to a closed configuration to secure the leaflet between the first jaw and the second jaw; bending the first jaw and the second jaw into a first position such that the first jaw and the second jaw are curved into a first curve; actuating the first cutter such that it moves along a first curved path following the first curve to cut the leaflet along a first cut following the first curved path; bending the first jaw and the second jaw into a second position different that the first position such that the first jaw and the second jaw are curved into a second curve; and actuating the second cutter such that it moves along a second curved path following the second curve to cut the leaflet along a second cut following the first second path.

In a twelfth example, in the method of the eleventh example, the steerable leaflet resection device further includes an anchoring tip extending between distal ends of the first and second jaws, wherein closing the first and second jaws to the closed configuration causes the anchoring tip to pinch the leaflet to secure the leaflet between the first and second jaws.

In a thirteenth example, in the method of the eleventh or twelfth example, bending the first jaw and the second jaw into the first position occurs before closing the first jaw and the second jaw to secure the leaflet therebetween.

In a fourteenth example, in the method of the eleventh or twelfth example, bending the first jaw and the second jaw into the first position occurs after closing the first jaw and the second jaw to secure the leaflet therebetween.

In a fifteenth example, in the method of any of the eleventh through fourteenth examples, bending the first jaw and the second jaw into the first position comprises pulling steering wires coupled to first sides of the first jaw and the second jaw.

In a sixteenth example, in the method of the fifteenth example, bending the first jaw and the second jaw into the second position comprises pulling steering wires coupled to second sides of the first jaw and the second jaw.

In a seventeenth example, in the method of any of the eleventh through sixteenth examples, the valve of interest is a native valve or a previously implanted transcatheter valve prosthesis or a previously implanted surgical valve prosthesis.

In an eighteenth example, in the method of any of the eleventh through seventeenth examples, the steerable leaflet resection device is configured to remove at least 50% of the leaflet, or at least 60% of the leaflet, or at least 70% of the leaflet, or at least 80% of the leaflet.

In a nineteenth example, in the method of any of the eleventh through eighteenth examples, the method further includes retracting the steerable leaflet resection device in the closed position with a resected portion of the leaflet disposed between the first and second jaws in the closed position after making the second cut.

In a twentieth example, in the method of any of the eleventh through nineteenth examples, the steerable leaflet resection device is tracked to the valve of interest with the first and second jaws in the closed configuration.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

The foregoing and other features and advantages of the present disclosure will be apparent from the following description of embodiments hereof as illustrated in the accompanying drawings. The accompanying drawings, which are incorporated herein and form a part of the specification, further serve to explain the principles of the present disclosure and to enable a person skilled in the pertinent art to make and use the embodiments of the present disclosure. The drawings may not be to scale.

FIG. 1A shows a side view of an example transcatheter heart valve prosthesis.

FIG. 1B shows a top view of the transcatheter heart valve prosthesis of FIG. 1A.

FIG. 1C shows the transcatheter heart valve prosthesis of FIG. 1A within deployed within a native aortic valve.

FIG. 2A shows a perspective side view of a distal portion of steerable leaflet resection device according to embodiments hereof.

FIG. 2B shows a close-up view of a distal end of the steerable leaflet resection device of FIG. 2A.

FIG. 2C shows a cross-section of the distal end of the steerable leaflet resection device if FIG. 2B.

FIG. 3A shows a side view of the distal portion of the steerable leaflet resection device in a closed configuration.

FIG. 3B shows a side view of the distal portion of the steerable leaflet resection device in an open configuration.

FIG. 4A shows a schematic illustration of the steerable leaflet resection device in a straightened configuration.

FIG. 4B shows a schematic illustration of the steerable leaflet resection device when steered in a first direction.

FIG. 4C shows a schematic illustration of a force diagram of the steerable leaflet resection device when cutting a portion of a leaflet.

FIG. 5 shows a schematic illustration of the steerable leaflet resection device in the closed configuration around a leaflet.

FIG. 6 is a block diagram that shows a method of using the steerable leaflet resection device according to embodiments hereof.

FIG. 7A shows a step in the method of FIG. 6, showing a schematic illustration of a side cross-sectional view of the distal portion of steerable leaflet resection device in the closed configuration around a leaflet and in a first position.

FIG. 7B shows a schematic illustration of a top view of the steerable leaflet resection device shown in FIG. 7A.

FIG. 7C shows a step in the method of FIG. 6, showing a schematic illustration of a side cross-sectional view of the steerable leaflet resection device making a first cut while in the first position.

FIG. 7D shows a schematic illustration of a top view of the steerable leaflet resection device shown in FIG. 7C.

FIG. 7E shows a step in the method of FIG. 6, showing a schematic illustration of the steerable leaflet resection device in a second position after being steered to the second position from the first position.

FIG. 7F shows a step in the method of FIG. 6, showing a schematic illustration of a side cross-sectional view of the steerable leaflet resection device making a second cut at the second position.

FIG. 7G shows a top view of the steerable leaflet resection device shown in FIG. 7F.

FIG. 7H shows a step in the method of FIG. 6, showing a top view of the leaflet with a resected portion of the leaflet removed from the leaflet.

FIG. 8A shows a side view of a jack mechanism in an open configuration according to embodiments hereof.

FIG. 8B shows a side view of the jack mechanism of FIG. 8A in the closed configuration according to embodiments hereof.

FIG. 9A shows a side view of a jack mechanism in an open configuration according to embodiments hereof.

FIG. 9B shows a side view of the jack mechanism of FIG. 9A in the closed configuration according to embodiments hereof.

### DETAILED DESCRIPTION

It should be understood that various embodiments disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single device or component for purposes of clarity, it should be understood that the techniques of this disclosure may be performed by a combination of devices or components associated with, for example, a delivery device. The following detailed description is merely exemplary in nature and is not intended to limit the invention of the application and uses of the invention. Furthermore, there is no intention to be bound by any expressed or implied theory presented in the preceding field of the invention, background, summary or the following detailed description.

As used in this specification, the singular forms “a”, “an” and “the” specifically also encompass the plural forms of the terms to which they refer, unless the content clearly dictates otherwise. The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%. It should be understood that use of the term “about” also includes the specifically recited number of value.

The terms “proximal” and “distal” herein are used with reference to the clinician using the devices. Therefore, “proximal” and “proximally” mean in the direction toward the clinician, and “distal” and “distally” mean in the direction away from the clinician.

Further, numerical terms such as “first”, “second”, “third”, etc. used herein are not meant to be limiting such that use of the term “second” when referring to a part in the specification does not mean that there necessarily is a “first” of part in order to fall within the scope of the invention. Instead, such numbers are merely describing that the particular embodiment being described has a “first” part and a “second” part. The invention is instead defined by the claims, in which one or more of the numbered parts may be claimed.

Further, terms such a “top” or “bottom” are used herein for the perspective in the drawings and ease of understanding, and are not meant to be limiting. Therefore, if a part, relationship between parts, or a view is referred to as the “top” or “bottom”, it is understood that these could be reversed, or the “top” and/or “bottom” could be sides of the part, the parts could be side-by-side, or it could be a side view.

Embodiments hereof relate to a steerable leaflet resection or cutting device or tool configured to resect one or more leaflets of a native heart valve and/or one or more leaflets of a previously implanted transcatheter or surgical heart valve prosthesis when implanting a transcatheter valve prosthesis. More particularly, in some embodiments, the steerable leaflet resection device is configured to cut a portion of the leaflet in a curved path such that a portion of the leaflet of a leaflet of a native heart valve or a previously implanted heart valve prosthesis is resected or removed prior to implantation of the additional transcatheter valve prosthesis. In some embodiments, the steerable leaflet resection device is configured to remove at least 40% of the leaflet, at least 50% of the leaflet, or at least 60% of the leaflet, or at least 70% of the leaflet. The actual amount of the leaflet that is resected is dependent on various factors, including the size of the target leaflet, the crossing profile of the catheter, and placement accuracy during the procedure.

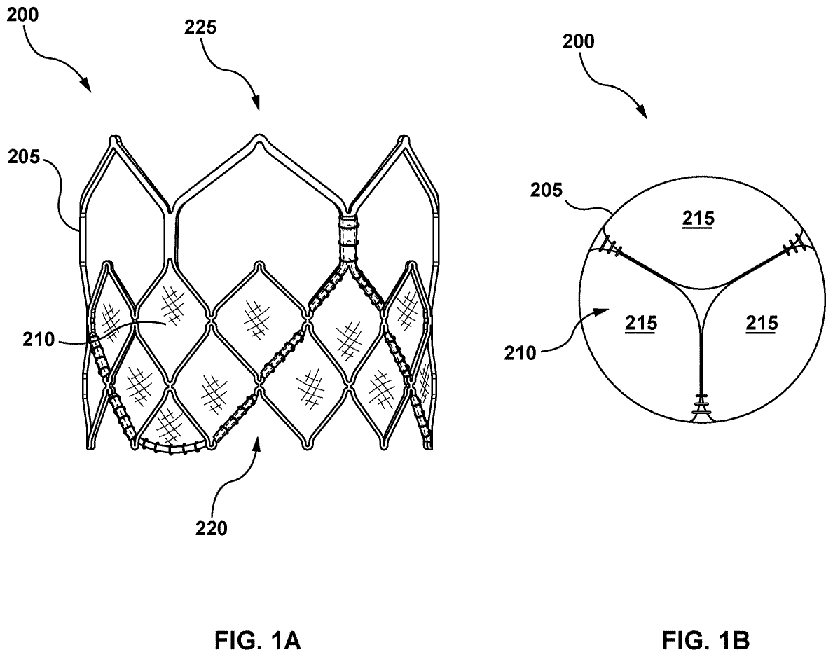

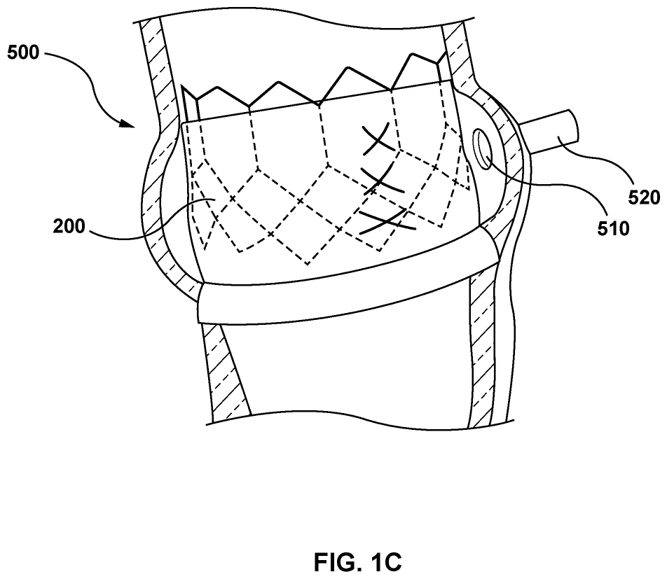

FIGS. 1A and 1B illustrate an example transcatheter heart valve prosthesis 200. The steerable leaflet resection device or tool described herein may be used to resect a portion of one or more of the leaflets of the transcatheter heart valve prosthesis 200 after the transcatheter heart valve prosthesis 200 has been implanted into a native valve or the steerable leaflet resection device or tool described herein may be used to resect a native valve leaflet prior to implantation of a transcatheter heart valve prosthesis such as the transcatheter heart valve prosthesis 200. The transcatheter heart valve prosthesis 200 is illustrated herein in order to facilitate description of the present invention. The following description of the transcatheter heart valve prosthesis 200 is merely exemplary in nature and is not intended to limit the invention or the application and uses of the invention. Although the transcatheter heart valve prosthesis 200 is a balloon-expandable heart valve prosthesis configured for placement within an aortic heart valve, embodiments of the steerable leaflet resection device described herein may be utilized to resect one or more leaflets of any transcatheter valve prosthesis. For example, and not by way of limitation, the embodiments of the steerable leaflet resection device or tool described herein may be utilized to resect one or more leaflets of a surgically implanted heart valve prosthesis, or a self-expanding transcatheter heart valve prosthesis. Further, although described herein with respect to a native aortic valve or a transcatheter aortic valve prosthesis, this is not mean to be limiting, and the steerable leaflet resection device describe herein can be used to resect one or more leaflets of a previously implanted valve or the native valve at the native pulmonary, aortic, mitral, or tricuspid valve, or may be utilized with a transcatheter valve prosthesis where it is deemed useful. There is no intention to be bound by any expressed or implied theory presented in the preceding technical field, background, brief summary or the following detailed description.

FIGS. 1A and 1B illustrate a side view and a top (outflow end) view, respectively, of the transcatheter heart valve prosthesis 200. The transcatheter heart valve prosthesis 200 includes a radially expandable frame or stent 205 and a prosthetic valve 210. The frame 205 of the transcatheter heart valve prosthesis 200 supports the prosthetic valve 210 within the interior of the frame 205. In the example transcatheter heart valve prosthesis shown in FIGS. 1A-1C, the frame 205 is balloon-expandable. However, this is not meant to be limiting, as the frame 205 can be self-expanding or otherwise mechanically expandable.

The prosthetic valve 210 includes at least one leaflet 215 disposed within and secured to the frame 205. In the embodiment shown in FIGS. 1A-1C, the prosthetic valve 210 includes exactly three leaflets 215, as best shown in FIG. 1B. However, this is not meant to be limiting, and the prosthetic valve 210 may include more or fewer leaflets 215. The valve leaflets 215 open and close to regulate flow through the transcatheter heart valve prosthesis 200.

As shown in FIG. 1A, the transcatheter heart valve prosthesis 200 includes an inflow end 220 and an outflow end 225. The prosthetic leaflets 215 are attached to the frame 205 such that when pressure at the inflow end 220 exceeds pressure at the outflow end 225, the prosthetic leaflets 215 open to allow blood flow through the heart valve prosthesis 200 from the inflow end 220 to the outflow end 225. When the pressure at the outflow end 225 exceeds pressure at the inflow end 220, the prosthetic leaflets 215 close to prevent blood flow from the outflow end 225 to the inflow end 220.

FIG. 1C shows the transcatheter heart valve prosthesis within a native aortic valve 500 of a patient. As can be seen, the transcatheter valve prosthesis 200 may be disposed adjacent to coronary ostia, one coronary ostium 510 being shown in FIG. 1C. The coronary ostium 510 is the opening of a coronary artery 520 disposed in the sinus of the native aortic valve 500 of the patient. In some cases, a second transcatheter heart valve prosthesis needs to be implanted within the transcatheter heart valve prosthesis 200 (i.e., a valve-in-valve procedure). In such a case, the second transcatheter heart valve prosthesis is inserted within the leaflets 215 of the transcatheter heart valve prosthesis 200 such that the leaflets 215 are opened and pressed against the frame 205. In such a case, the leaflets 215 of the transcatheter heart valve prosthesis 200 may block the coronary ostia 210. This blocking may also occur when implanting a transcatheter heart valve prosthesis in a previously installed surgical valve. Therefore, prior to installing a second transcatheter heart valve prosthesis in the previously implanted transcatheter heart valve prosthesis 200 or in a surgically installed heart valve prosthesis, it is desirable to resect or remove at least a portion of one or more of the leaflets of the previously implanted device. Further, in some instances, it may be desirable to resect or remove native valve leaflets prior to implantation of a first transcatheter heart valve prosthesis. The present application is directed to a leaflet resection device to accomplish such resection.

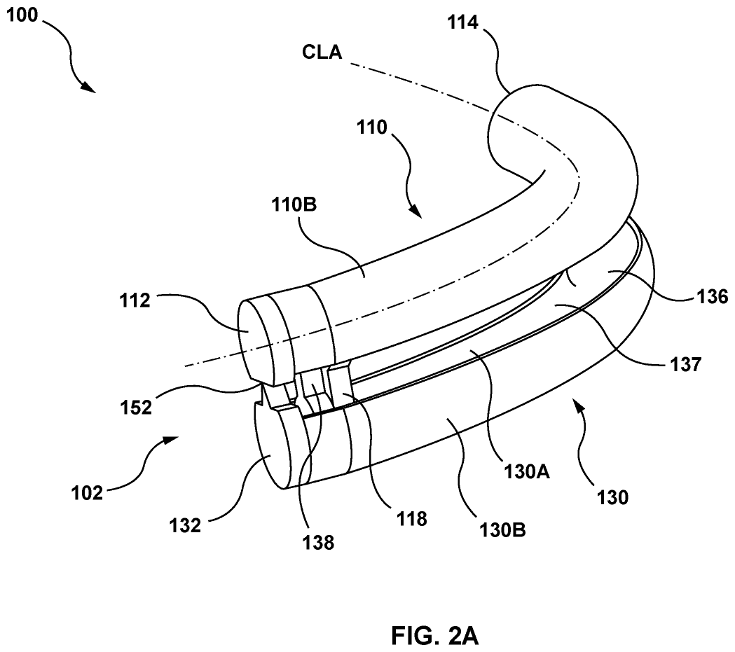

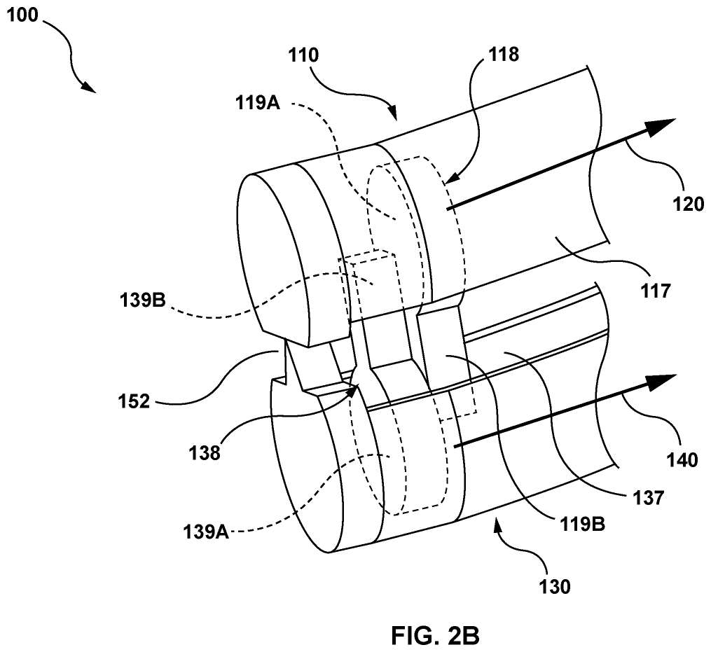

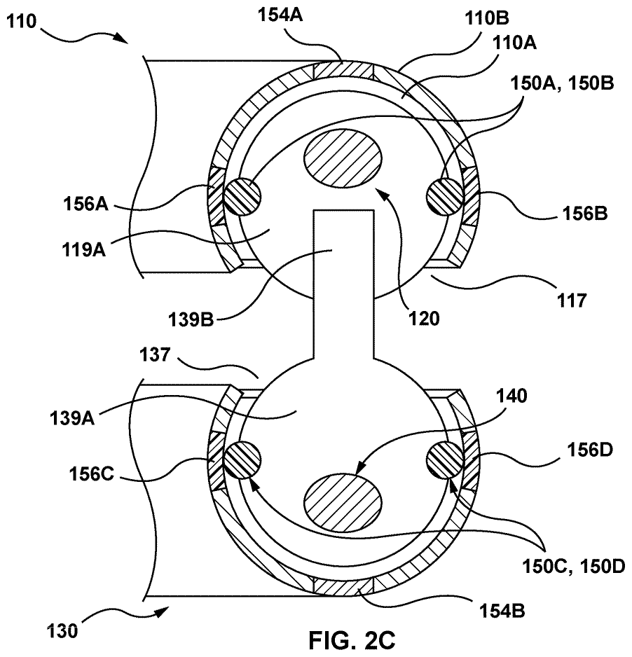

FIGS. 2A-2C show a distal portion of a steerable leaflet resection device 100 according to embodiments herein. The steerable leaflet resection device 100 is includes a distal end 102 and a proximal end (not shown). As noted, only the distal portion of the steerable leaflet resection device 100. The components noted extend proximally outside of the body to the proximal end (not shown) which includes a handle (not shown) including actuators (not shown) configured to operates the components as explained below, as would be understood by those skilled in the art. One skilled in the art will realize that FIGS. 2A-2C illustrate one example of a steerable leaflet resection device and that existing components illustrated in FIGS. 2A-2C may be removed and/or additional components may be added to the steerable leaflet resection device 100.

As shown, the distal portion of the steerable leaflet resection device 100 includes a first jaw 110, a second jaw 130 connected to the first jaw 110 via a jack mechanism 160, a first blade 118 and a first pullwire 120 coupled to the first jaw 110, and a second blade 138 and a second pullwire 140 coupled to the second jaw 130.

As can be seen in FIGS. 2B and 2C, the first jaw 110 is a hollow, tube-shaped member, wherein a cross-section of the first jaw 110 is substantially semi-circular shaped, as shown best in FIG. 2C. When viewed from the distal end 102 of the steerable leaflet resection device 100 (FIG. 2C), the first jaw 110 concaves down such that a bottom portion of the first jaw 110 is discontinuous and open. The first jaw 110 includes a distal end 112 a proximal end 114. The first jaw 110 further includes an outer surface 110B and an inner surface 110A defining a cavity or space 116 within the inner surface 110A of the first jaw 110. The distal end 112 of the first jaw 110 may include a flat surface or cap that encloses the cavity 116 of the first jaw 110 such that the cavity 116 of the first jaw 110 is not exposed at the distal end 112 of the first jaw 110. In other words, the flat surface or cap at the distal end 112 of the first jaw 110 prevents the cavity 116 from extending through the distal end 112 of the first jaw 110 such that the cavity 116 has a closed distal end, as shown in FIG. 2A. The discontinuous wall of the first jaw 110 creates a longitudinal groove 117 at the bottom of the first jaw 110, facing the second jaw 130. The first jaw 110 has a longitudinal length of about 10-30 mm and a diameter of about 2-5 mm, the length being defined from the jack mechanism 160 described below to the distal end 112 of the first jaw 110, i.e. from the location where the first jaw 110 is coupled to the second jaw 130 to the distal end 112 of the first jaw 110. The first jaw 110 can comprise various flexible and stiff materials, which will be described in further detail below.

The second jaw 130 is a hollow tube-shaped member, wherein a cross-section of the second jaw 130 is substantially semi-circular shaped, as shown best in FIG. 2C. When viewed from the distal end 102 of the steerable leaflet resection device 100 (FIG. 2C), the second jaw 130 concaves up such that a top portion of the second jaw 130 is discontinuous and open. The second jaw 130 includes a distal end 132 and a proximal end 134. The second jaw 130 further includes an outer surface 130B and an inner surface 130A defining a cavity or space 136 within the inner surface 130A of the first jaw 130. The distal end 132 of the second jaw 130 may include a flat surface or cap that encloses the cavity 136 of the second jaw 130 such that the cavity 136 of the second jaw 130 is not exposed at the distal end 132 of the second jaw 130. In other words, the flat surface or cap at the distal end 132 of the second jaw 130 prevents the cavity 136 from extending through the distal end 132 of the second jaw 130 such that the cavity 136 has a closed distal end, as shown in FIG. 2A. The discontinuous wall of the second jaw 130 creates a longitudinal groove 137 at the top of the second jaw 130, facing the first jaw 110. The second jaw 130 has a longitudinal length of about 10-30 mm and a diameter of about 2-5 mm, the length being defined from the jack mechanism 160 described below to the distal end 132 of the second jaw 130, i.e. from the location where the second jaw 130 is coupled to the first jaw 110 to the distal end 132 of the second jaw 130. The second jaw 130 can comprise various flexible and stiff materials, which will be described in further detail below.

The proximal ends 114, 134 of the first and second jaws 110, 130 are coupled together via a jack mechanism 160, which will be described in more detail below. The jack mechanism 160 enables the first and second jaws 110, 130 to open and close with respect to each other. The jack mechanism 160 can be a hinge connection, a hydraulic powered jack and/or any other jack mechanism configurations known to those skilled in the art.

As can be seen in FIG. 2B, the distal end 132 of the second jaw 130 further includes an anchoring tip 152. The anchoring tip 152 includes a stiff, sharp point that extends towards the first jaw 110 (vertically upwards in the drawings). When the steerable leaflet resection device 100 is in a closed configuration, which will be described in further detail below, the point of the anchoring tip 152 contacts or abuts the distal end 112 of the first jaw 110, as can be seen in FIG. 2B. However, this is not meant to be limiting in that the anchoring tip 152 need not extend completely to the first jaw 110. Further, instead of the anchoring tip 152 extending from the second jaw 130 to the first jaw 110, the anchoring tip 152 could extend from the first jaw 110 to the second jaw 130. The anchoring tip 152 is configured to secure the first jaw 110 and the second jaw 130 onto a leaflet 215 and act as a pivot point for the steerable leaflet resection device 100, which will be described in more detail below. The anchoring tip 152 can have a height of about 0.1-2.0 mm and can comprise a rigid metal such as stainless steel, a rigid polymer such as polyetheretherketone (PEEK) and/or any combination of materials known to those skilled in the art. A rigid material is desirable for the anchoring tip 152 in order to embed in the leaflet tissue. Additionally, the anchoring tip 152 may include a radiopaque marker or a polymer with metal additives so the tip location can be more easily visualized using fluoroscopy during the leaflet resection process.

The first jaw 110 further includes a first cutter 118 and a first pullwire 120, as can be seen in FIG. 2B. The first cutter 118 includes a first base 119A that has a distal side, a proximal side, and a substantially circular cross-section. The first base 119A is sized and shaped to slidably fit within the cavity 116 of the first jaw 110, as shown in FIG. 2B. When the first base 119A is situated within the cavity 116 of the first jaw 110, the distal side of the first base 119A faces the distal end 112 of the first jaw 110, and the proximal side of the first base 119A faces the proximal end 114 of the first jaw 110. A first blade 119B extends from the first base 119A out of the longitudinal groove 117 of the first jaw 110 and towards the second jaw 130. The first blade 119B includes a sharp tip 119C configured to pierce through leaflet tissue, and may also include sharp edges configured to cut leaflet tissue as the first blade 119B is moved relative to such leaflet tissue, as described below. The first pullwire 120 is coupled to the first base 119A and extends in a proximal direction towards the proximal end 114 of the first jaw 110, as can be seen in FIG. 2B. The first pullwire 120 is continues to extend proximally to an actuator (not shown) at a proximal end of the steerable leaflet resection device 100 such that actuation of the actuator pulls the first cutter 118 from the distal end 112 of the first jaw 110 to the proximal end 114 of the first jaw 110, which will be described in further detail below. The first base 119A can have a diameter of about 1-4 mm, a length of about 0.5-5.0 mm, and can comprise stainless steel, titanium and/or any other materials known to those skilled in the art. The first blade 119B can extend about 0.5-6.0 mm from the bottom of the first base 119A and can comprise stainless steel, titanium and/or any other materials known to those skilled in the art. The first pullwire 120 can comprise single filament or braided multi-filament wire made of, for example, stainless steel, or any other materials known to those skilled in the art.

The second jaw 130 further includes a second cutter 138 and a second pullwire 140, as can be seen in FIG. 2B. The second cutter 138 includes a second base 139A that has a distal side, a proximal side, and a substantially circular cross-section. The second base 139A is sized and shaped to slidably fit within the cavity 136 of the second jaw 130, as shown in FIG. 2B. When the second base 139A is situated within the cavity 136 of the second jaw 130, the distal side of the second base 139A faces the distal end 132 of the second jaw 130, and the proximal side of the second base 139A faces the proximal end 134 of the second jaw 130. A second blade 139B extends from the second base 139A out of the longitudinal groove 137 of the second jaw 130 and towards the first jaw 110. The second blade 139B includes a sharp tip 139C configured to pierce through leaflet tissue, and may also include sharp edges configured to cut leaflet tissue as the second blade 139B is moved relative to such leaflet tissue, as described below. The second pullwire 140 is coupled to the second base 139A and extends in a proximal direction towards the proximal end 134 of the second jaw 130, as can be seen in FIG. 2B. The second pullwire 140 is continues to extend proximally to an actuator (not shown) at a proximal end of the steerable leaflet resection device 100 such that actuation of the actuator pulls the second cutter 138 from the distal end 132 of the second jaw 130 to the proximal end 134 of the second jaw 130, which will be described in further detail below. The second base 139A can have a diameter of about 1-4 mm, a length of about 0.5-5.0 mm, and can comprise stainless steel, titanium and/or any other materials known to those skilled in the art. The second blade 139B can extend about 0.5-6.0 mm from the top of the second base 139A and can comprise stainless steel, titanium and/or any other materials known to those skilled in the art. The second pullwire 140 can comprise single filament or braided multi-filament wire made of for example, stainless steel, or any other materials known to those skilled in the art.

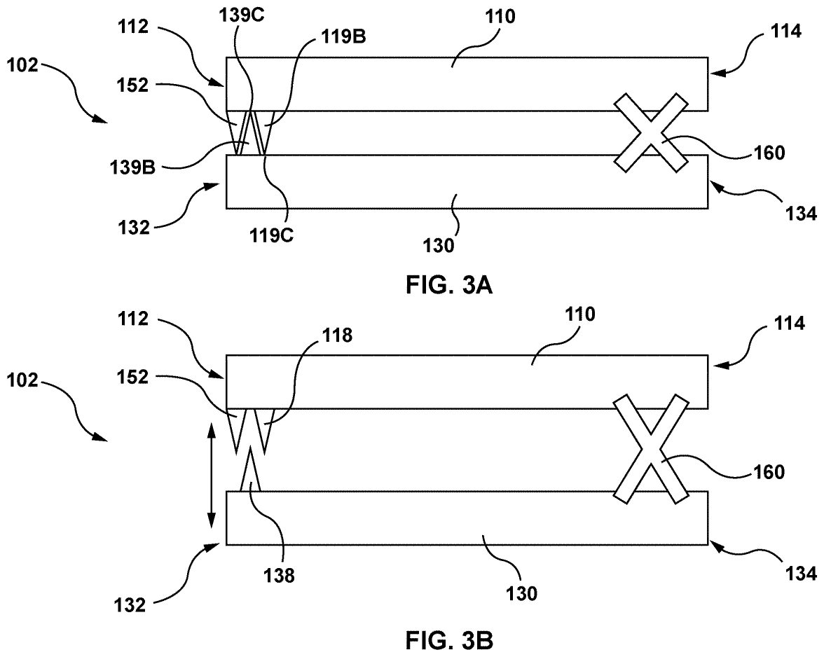

In a delivery configuration of the steerable leaflet resection device 100, the jaws 110, 130 of the steerable leaflet resection device 100 are in the closed configuration, as shown in FIGS. 2A-2B and 3A. In the closed configuration, the jaws 110, 130 are adjacent to each other, and the first cutter 118 and the second cutter 138 are disposed at the distal ends 112, 132 of the first and second jaws 110, 130, respectively. In the particular embodiment shown, the second base 139A and the second blade 139B are disposed distal to the first base 119A and the first blade 119B, such that the distal side of the first base 119A abuts the proximal side of the second base 139A. However, this is not meant to be limiting and either cutter may be disposed distal of the other cutter.

As described above and shown in FIGS. 2A-2B, the cavity 116 of the first jaw 110 houses the first cutter 118, including the first base 119A and the first blade 119B, and the first pullwire 120, and the cavity 136 of the second jaw 130 houses the second cutter 138, including the second base 139A and the second blade 139B, and the second pullwire 140. However, this is not meant to be limiting, and either of the first and second jaws 110, 130 may house both the first cutter 118 and the second cutter 138. In such embodiments, the first and second cutters 118, 138 are disposed such that they may be actuated separately, as explained in more detail below.

FIG. 2C shows a cross-section of the steerable leaflet resection device 100 of FIGS. 2A-2B when viewed from the front or distal end 102. As can be seen, in the orientation shown, the first jaw 110 is disposed vertically above the second jaw 130, but this is not meant to be limiting as the orientation of the device may be changed. The second base 139A and the second blade 13B are disposed distal of (in FIG. 2C, in front of) the first base 119A and the first blade 119B. Pullwires 120, 140 are coupled to a center portion of the first base 119A and the second base 139A, respectively.

Each of the first and second jaws 110, 130 further includes a rigid spine 154A, 154B, respectively. In the orientation with the first jaw 110 above the second jaw 130, the rigid spine 154A of the first jaw 110 is disposed at a top, center portion of the first jaw 110 and the rigid spine 154B of the second jaw 130B is disposed at a bottom, center portion of the second jaw 130, as shown in FIG. 2C. The rigid spines 154A, 154B extend an entire longitudinal length of the first and second jaws 110, 130, respectively, having a length of about 10-30 mm. The rigid spines 154A, 154B can have a width of about 1-8 mm and can comprise stainless steel, titanium and/or any other materials known to those skilled in the art. The rigid spines 154A, 154B are configured to maintain the stiffness of the first and second jaws 110, 130 of the steerable leaflet resection device 100 when the device 100 is being steered such that the steering is lateral with respect to the orientation shown and the vertical orientation of the steerable leaflet resection device 100 is maintained when the device 100 is being steered, which will be described in further detail below.

Each of the first and second jaws 110, 130 further includes two flexible regions 156A-156D, respectively. The first jaw 110 includes a first and a second flexible region 156A, 156B disposed at both lateral sides of the first jaw 110, approximately 90 degrees from the rigid spine 154A in both directions, as can be seen in FIG. 2C. The second jaw 130 includes a third and a fourth flexible region 156C, 156D disposed at both lateral sides of the second jaw 130, approximately 90 degrees from the rigid spine 154B in both directions, as can be seen in FIG. 2C. The flexible regions 156A-156D extend an entire longitudinal length of the first and second jaws 110, 130, respectively, having a length of about 10-30 mm. The flexible regions 156A-156D can have a width of about 1-8 mm and can comprise stainless steel, nitinol and/or any other materials known to those skilled in the art. The steerable leaflet resection device 100 further includes steering pullwires 150A-150D. The steering pullwires 150A-150D are coupled to the inner surfaces of each corresponding flexible region 156A-156D. More specifically, a first steering pullwire 150A is coupled to an inner surface of the first flexible region 156A of the first jaw 110 and a second steering pullwire 150B is coupled to an inner surface of the second flexible region 156B of the first jaw 110, as shown in FIG. 2C. Further, a third steering pullwire 150C is coupled to an inner surface of the third flexible region 156C of the second jaw 130, and a fourth steering pullwire 150D is coupled to an inner surface of the fourth flexible region 156D of the second jaw 130, as shown in FIG. 2C. The steering pullwires 150A-150D extend an entire longitudinal length of the first and second jaws 110, 130 and are continuously coupled to the first and second jaws 110, 130 along their entire length. Although the steering pullwires 150A-150D are coupled to the first and second jaws 110, 130 along their entire length, the steering pullwires 150A-150D remain free to slide relative to the first and second jaws 110, 130, as the jaws will shorten or elongate as they are bent and straightened. Further, the steering pullwires 150A-150D extend proximally to a handle of the steerable leaflet resection device 100 and are coupled to actuators (not shown) that enable a clinician to apply and release tension to (i.e. pull and release) the steering pullwires 150A-150D. The flexible regions 156A-156D and steering pullwires 150A-150D of the first and second jaws 110, 130 allow a user to steer the steerable leaflet resection device 100 to reach a desired cut path by pulling or tensioning some of the steering pullwires 150A-150D, as described below. The flexible regions 156A-156D of the first and second jaws 110, 130 allow the lateral sides of the first and second jaws 110, 130 to bend when the steering pullwires 150A-150D are tensioned, which will be described in further detail below. Each of the four flexible regions 156A-156D have a width of about 1-8 mm and comprise stainless steel, nitinol and/or any other material known to those skilled in the art.

FIGS. 3A-3B show schematic side cross-sectional views of the steerable leaflet resection device 100 in a closed configuration and an open configuration, respectively. As stated previously, the proximal ends 114, 134 of the first jaw 110 and the second jaw 130 are coupled to one another via a jack mechanism 160, as can be seen in FIGS. 3A-3B. In the closed configuration, the first jaw 110 and the second jaw 130 are disposed substantially parallel to one another. In the orientation shown, the first jaw 110 is disposed above of the second jaw 130 such that the first and second jaws 110, 130 overlap when viewed from above. In the embodiment shown in FIGS. 3A-3B, the anchoring tip 152 is disposed on the distal end 112 of the first jaw 110 and extends towards the second jaw 130, but as noted above, it could be the opposite.

The jack mechanism 160 is configured to convert the steerable leaflet resection device 100 from the closed configuration of FIG. 3A with the first and second jaws 110, 130 adjacent to each other to the open configuration of FIG. 3B with the first and second jaws 110, 130 separated from each other. In the embodiment shown, the jack mechanism 160 is actuated such that the first jaw 110 and the second jaw 130 are separated by moving the first jaw 110, the second jaw 130 or both jaws 110, 130. The jaws 110, 130 are separated until there is a desirable amount of space or distance between the point of the anchoring tip 152 and the tip 119C of the first blade 119B to the tip 139C of the second blade 139B, as shown in FIG. 3B. The desirable amount of space is enough to ensure a leaflet 215 will fit between the tips when the steerable leaflet resection device 100 is in the open configuration, which will be described in more detail below. Although the jack mechanism 160 is shown as separating the first and second jaws 110, 130 from the proximal to distal ends thereof, in other embodiments the jack mechanism 160 may act more like a hinge such that the distal ends 112, 132 of the first and second jaws 110, 130 separate from each other to create the space described above, but the proximal ends 114, 134 of the first and second jaws 110, 130 remain adjacent to each other, as known to those skilled in the art. To convert the steerable leaflet resection device 100 from the open configuration to the closed configuration, the jack mechanism 160 is actuated such that the first and second jaws 110, 130 move towards each, opposite of the opening described above.

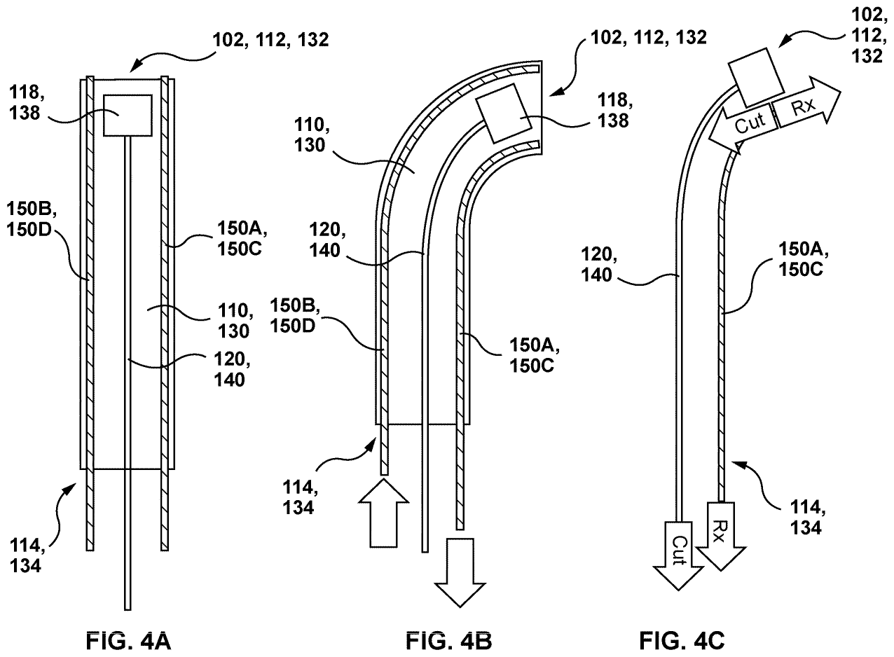

FIGS. 4A-4B are used to describe the steering mechanism of the steerable leaflet resection device 100. As can be seen, FIGS. 4A-4B are schematic illustrations of the steerable leaflet resection device 100 from a top view (above, looking down). The distal end 102 of the steerable leaflet resection device 100 and the distal ends 112, 132 of the first and second jaws 110, 130 are portrayed at the top of the figures and the proximal ends 114, 134 of the first and second jaws 110, 130 of the jaws are portrayed at the bottom of the figures, with the steerable leaflet resection device 100 continuing proximally (not shown). For simplicity purposes, the schematics of FIGS. 4A-4B represent both the first jaw 110 and the second jaw 130 of the steerable leaflet resection device 100, as their components overlap or mirror one another when viewed from above in the orientation shown. FIG. 4A shows the steerable leaflet resection device 100 in a non-tensioned configuration. The steerable leaflet resection device 100 is in the non-tensioned configuration when the four steering pullwires 150A-150D are not being pulled to create tension. Thus, in the non-tensioned configuration, there are no forces acting upon the four steering pullwires 150A-150D such that all four steering pullwires 150A-150D are relaxed and the first and second jaws 110, 130 of the steerable leaflet resection device 100 are substantially straight, as can be seen in FIG. 4A. Those skilled in the art would recognize that the steerable leaflet resection device 100 in the non-tensioned configuration can bend, such as to follow the curvature of a vessel, but it is not actively steered or curved.

To steer the steerable leaflet resection device 100 in a desired direction, the steering pullwires on the same side of both the first and second jaws 110, 130 are be pulled while the steering pullwires on the opposite side remain without tension. For example, if the user would like to steer the steerable leaflet resection device 100 to the right, as shown in FIG. 4B, the steering pullwires 150A, 150C on the right side of the steerable leaflet resection device 100 are pulled back proximally while the steering pullwires 150B, 150D on the left side of the steerable leaflet resection device 100 remain untensioned. More specifically, the steering wire 150A on the right side of the first jaw 110 and the steering wire 150C on the right side of the second jaw 130 are pulled. Pulling the steering wires 150A, 150C back proximally forces the first and second jaws 110, 130 to bend such that the distal end 102 of the steerable leaflet resection device 100 begins to face the right, as can be seen in FIG. 4B. The flexible regions 156A-156D of the first and second jaws 110, 130 allow the first and second jaws 110, 130 to bend laterally when tension is applied to the right-side steering pullwires 150A, 150C of the steerable leaflet resection device 100. In addition to steering the leaflet resection device 100, the bends in the first and second jaws 110, 130 enable the first and second cutters 118, 138 to follow a curved path when actuated, as explained in more detail below.

FIG. 4C shows a force diagram of the right-side pullwires 150A, 150C of the steerable leaflet resection device 100 when the steerable leaflet resection device 100 is being bent to the right and one of the cutters 118, 138 is being retracted proximally to cut a leaflet, as described in more detail below. In the example shown, the right-side steering pullwires 150A, 150C of the first and second jaws 110, 130 are pulled proximally to steer or bend the jaws 110, 130 to the right, as shown in FIG. 4C. When the right-side pullwires 150A, 150C are pulled and held under tension, the right-side pullwires provide a tension force proximal and distally, as shown in by the Rx arrows in FIG. 4C. This provides a stable position such that the first pullwire 120 or the second pullwire 140 for the first or second cutters 118, 138, respectively, may be pulled proximally to retract the first or second cutter 118, 138 to cut the leaflet. In other words, an advantage of applying tension to one side of the steerable leaflet resection device 100 is that it allows the steerable leaflet resection device 100 to be held securely in place when it is time to make a cut through a leaflet, which will be described in further detail below.

Similarly, if the user would like to steer the steerable leaflet resection device 100 to the left or bend the jaws 110, 130 to the left for a curved cutting path, as described below, the left-side steering pullwires 150B, 150D of the steerable leaflet resection device 100 are be pulled back proximally while the right-side steering pullwires 150A, 150C of the steerable leaflet resection device 100 remain untensioned. More specifically, the steering pullwire 150B on the left side of the first jaw 110 and the steering pullwire 150D on the left side of the second jaw 130 are pulled to steer the steerable leaflet resection device 100 to the left. Pulling the left-side steering pullwires 150B, 150D back proximally forces the first and second jaws 110, 130 to bend such that the distal end 102 of the steerable leaflet resection device 100 begins to face the left.

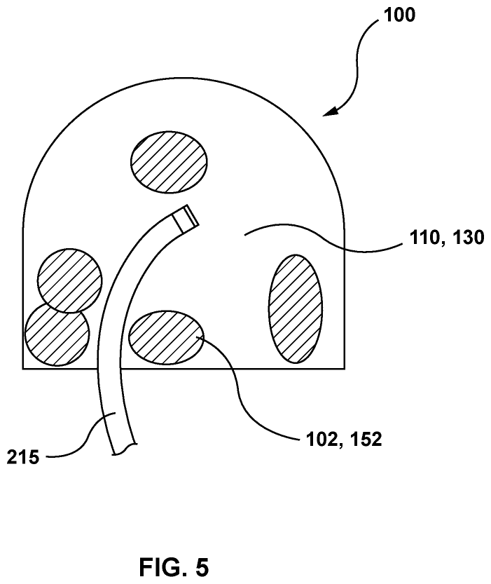

The steerable leaflet resection device 100 described herein is configured to be tracked to a desired location within the patient's vasculature in the untensioned configuration and also allow a user to steer the device 100 around calcification spots and other areas to avoid on a leaflet that is to be resected. FIG. 5 shows an exemplary leaflet 215 with calcium deposits throughout the leaflet 215. The steerable leaflet resection device 100 described herein is configured to allow a user to steer around the calcification spots of the leaflet 215 and still be able to resect a portion of the leaflet 215, which will be described in further detail below.

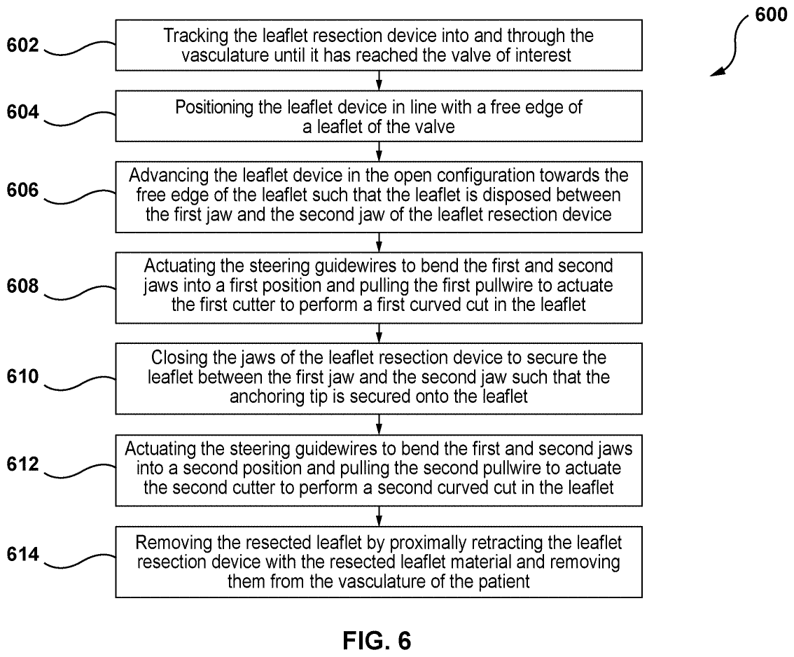

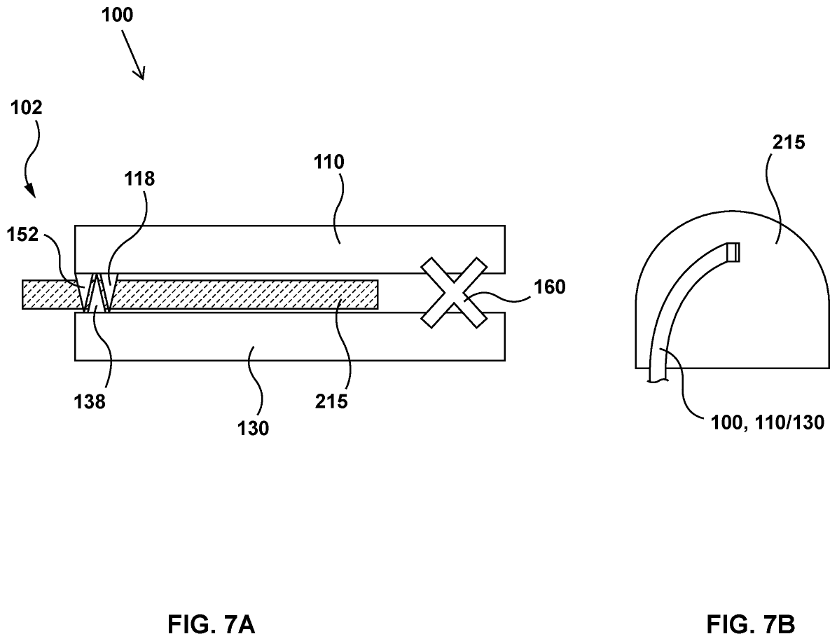

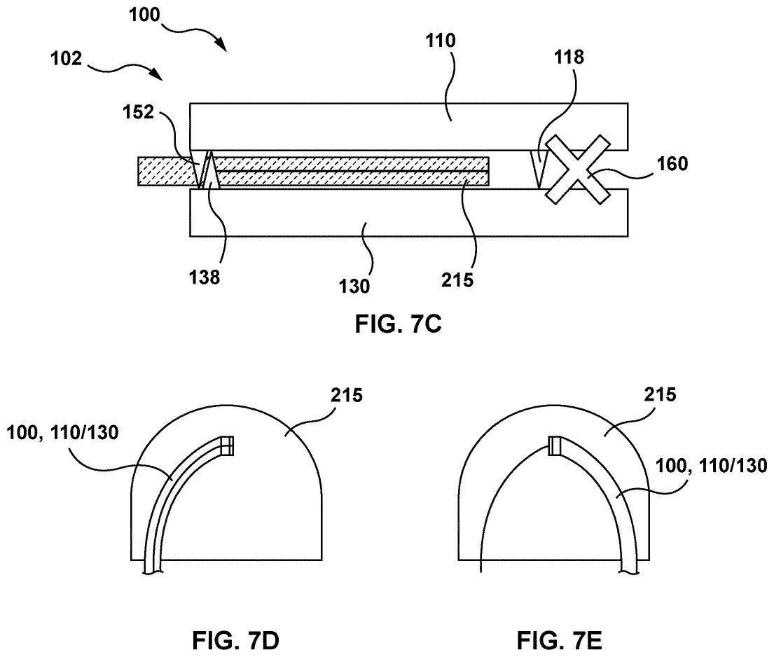

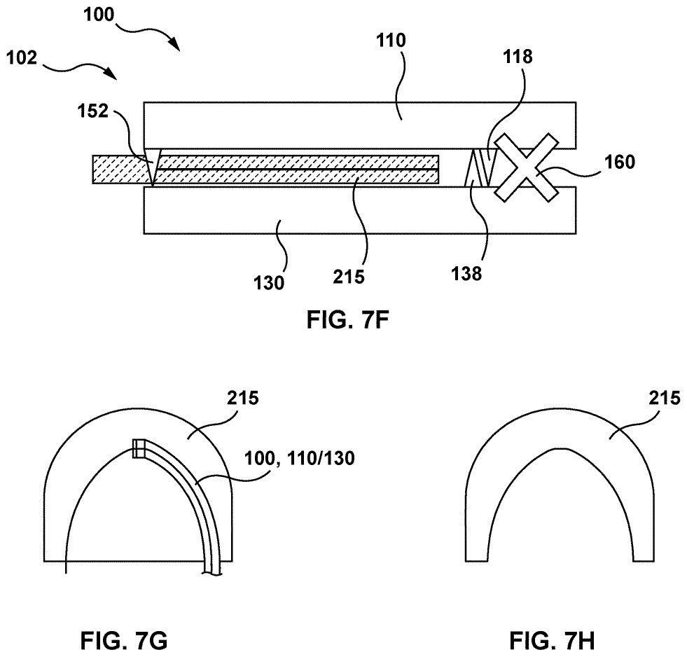

FIGS. 6-7H illustrate a method for resecting a leaflet of a valve of interest within a patient using the steerable leaflet resection device 100 according to embodiments herein. As previously discussed above, the valve of interest can be, for example, a native heart valve or a previously-implanted heart valve prosthesis. Accordingly, the leaflet to be resected can be, for example, a native heart valve leaflet or a prosthetic leaflet from the previously implanted heart valve prosthesis. In the embodiments described herein, the leaflet to be resected is a leaflet 215 of a previously implanted transcatheter heart valve prosthesis 200, but this only used as an example for identification purposes and the leaflet 215 can instead be any of the leaflets described above.

In a step 602 of the method (not shown), the steerable leaflet resection device 100 is tracked into and through the vasculature to the valve of interest. For example, and not by way of limitation, the valve of interest can be the native aortic valve or a previously implanted valve prosthesis at the native aortic valve. The steerable leaflet resection device 100 may be tracked into the vasculature by techniques known to those skilled in the art, such as the Seldinger technique. Further, the steerable leaflet resection device 100 may be tracked through the vasculature to the valve of interest by techniques known to those skilled in the art. The steerable leaflet resection device 100 is tracked to the valve of interested with the jaws 110, 130 in the closed position.

In a step 604 of the method 600, the steerable leaflet resection device 100 is positioned in line with a free edge of the leaflet 215 of the valve of interest. In a step 606 of the method 600, the jack mechanism 160 is actuated such that the jaws 110, 130 of the steerable leaflet resection 100 transition from the closed configuration to the open configuration. With the jaws 110, 130 in the open configuration, the steerable leaflet resection device 100 is advanced towards the free edge of the leaflet 215 such that a center portion of the leaflet 215 is disposed between the first jaw 110 and the second jaw 130 of the steerable leaflet resection device 100. If there are any calcium deposits on the leaflet 215, for example, the steerable leaflet resection device 100 may be steered around such calcium deposits using the steering pullwires 150.

In a step 608 of the method 600, the steerable leaflet resection device 100 is moved to a first position via the steering pullwires 150. As shown in FIG. 7B, the right-side pullwires 150A, 150C are pulled proximally such that the right side of the first and second jaws 110, 130 bend under tension. The tension on the right side of the steerable leaflet resection device 100 cause the first and second jaws 110, 130 of the steerable leaflet resection device 100 to bend radially outward to the left of the anchoring tip 152 such that the distal end 102 of the steerable leaflet resection device 100 faces to the right at the anchoring tip 152, as shown in FIG. 7B. The first position is a curved configuration of the first and second jaws 110, 130. When the first and second jaws 110, 130 are closed and the anchoring tip 152 is secured to the leaflet 215, the increase in bend angle will cause the jaws 110, 130 to shift towards the margin of attachment (MOA) of the leaflet. The degree of steering can be adjusted to better match the contour of the MOA or maximize the resection area of the leaflet 215.

In a step 610 of the method 600, once the steerable leaflet resection device 100 device 100 is in the desired position around the leaflet 215, the jack mechanism 160 is actuated to transition the jaws 110, 130 from the open configuration to the closed configuration around the leaflet 215, as shown in FIGS. 7A-7B. When the jaws 110, 130 are in the closed configuration around the leaflet 215, the anchoring tip 152 disposed at the distal end 112 of the first jaw 110 pinches the leaflet 215 in between the point of the anchoring tip 152 and the distal end 132 of the second jaw 130, as shown in FIG. 7A. The anchoring tip 152 does not need to puncture through the leaflet 215, however the first and second blades 118, 138 of the steerable leaflet resection device 100 must puncture through the leaflet 215 in the closed configuration. The anchoring tip 152 acts as a pivot point for the steerable leaflet resection device 100, as the anchoring tip 152 remains secured to the leaflet 215 during the entire resection process, which will be described further below. Although step 610 is described herein after step 608, it would be understood by those skilled in the art that the order could be reversed. In other words, the jaws 110, 130 may be closed prior to bending the steerable leaflet resection device 100 into the first position.

Once the steerable leaflet resection device 100 is in the first position shown in FIG. 7B with the jaws 110, 130 closed, the steerable leaflet resection device 100 is ready to make a first cut through the leaflet 215. To do this, the first pullwire 120 of the first cutter 118 is pulled back proximally such that the first base 119A is pulled back proximally within the cavity 116 of the first jaw 110, thereby also pulling first blade 119B proximally, as shown in FIGS. 7C-7D. As the first pullwire 120 is pulled back towards the proximal end 114 of the first jaw 110, the first blade 119B creates a first cut through the leaflet 215, starting at or near the anchoring tip 152, with the first cut following the bend or orientation of the first and second jaws 110, 130 of the steerable leaflet resection device 100, as shown in FIG. 7D. As shown, the first cut is a curved cut. The first pullwire 120 pulls the first cutter 118 back proximally until the first blade 119B cuts through the free-edge of the leaflet 215, as shown in FIGS. 7D-7E. The anchoring tip 152 and the second blade 138 of the steerable leaflet resection device 100 remain in the same position during this step, as shown in FIG. 7C.

In a step 612 of the method 600, after the first cut using the first cutter 118 of the steerable leaflet device 100 is complete, the steerable leaflet resection device 100 is moved to a second position via the steering pullwires 150. As shown in FIG. 7E, the left-side pullwires 150B, 150D are pulled proximally such that the left side of the first and second jaws 110, 130 bend under tension. The tension on the left side of the steerable leaflet resection device 100 cause the first and second jaws 110, 130 of the steerable leaflet resection device 100 to bend radially outward to the right of the anchoring tip 152 such that the distal end 102 of the steerable leaflet resection device 100 faces to the left at the anchoring tip 152, as shown in FIG. 7E. Thus, as can be seen in comparing FIG. 7D to FIG. 7B, the bend in the first and second jaws 110, 130 is essentially opposite or a mirror image in the second position as compared to the first position.

Once the steerable leaflet resection device 100 is in the second position shown in FIG. 7E, the steerable leaflet resection device 100 is ready to make a second cut through the leaflet 215. To do this, the second pullwire 140 of the second cutter 138 is pulled back proximally such that the second base 139A is pulled back proximally within the cavity 136 of the second jaw 130, thereby also pulling the second blade 139B, as shown in FIGS. 7F-7G. As the second pullwire 140 is pulled back towards the proximal end 134 of the second jaw 130, the second blade 139B creates a second cut through the leaflet 215, starting at or near the anchoring tip 152, that follows the bend or orientation of the first and second jaws 110, 130 of the steerable leaflet resection device 100, as shown in FIG. 7G. The second pullwire 140 pulls the second blade 138 back proximally until the second blade 138 cuts through the free-edge of the leaflet 215, as shown in FIGS. 7G-7H. At this step, the resected portion of the leaflet 215 is disconnected from the remaining portion of the leaflet 215 and can be removed. The jaws 110, 130 remain in the closed position such that the first and second jaws 110, 130 contact and capture a portion of the resected leaflet between the jaws 110, 130.

In a step 614 of the method 600 (not shown), the steerable leaflet resection device 100 can be retracted proximally from the leaflet 215 in the closed configuration with the resected portion of the leaflet 215 disposed between the first and second jaws 110, 130 of the steerable leaflet resection device 100. The steerable leaflet resection device 100 may retracted into a sheath or sleeve, such as guide catheter, adjacent the valve of interest such that the resected portion of the leaflet does not pull away from between the first and second jaws 110, 130 while removing the steerable leaflet resection device 100 from the vasculature of the patient.

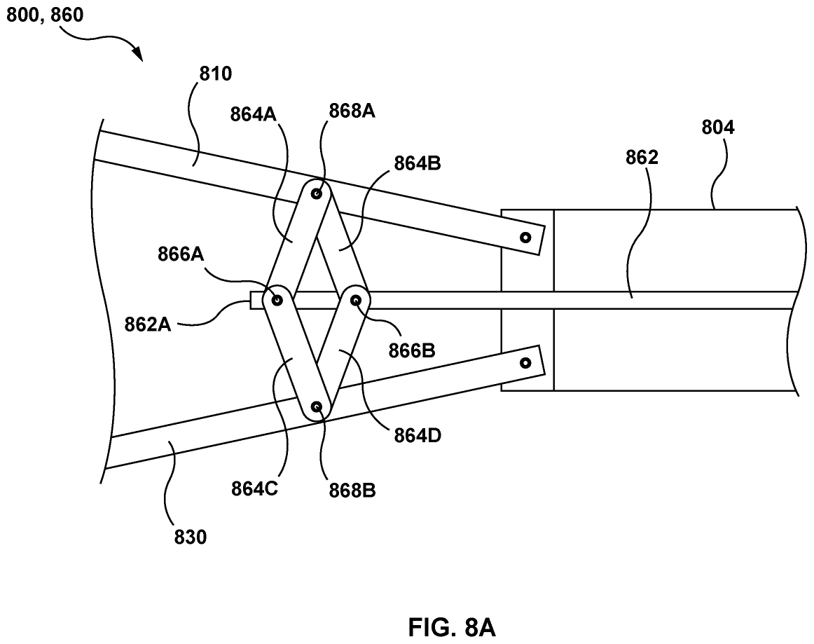

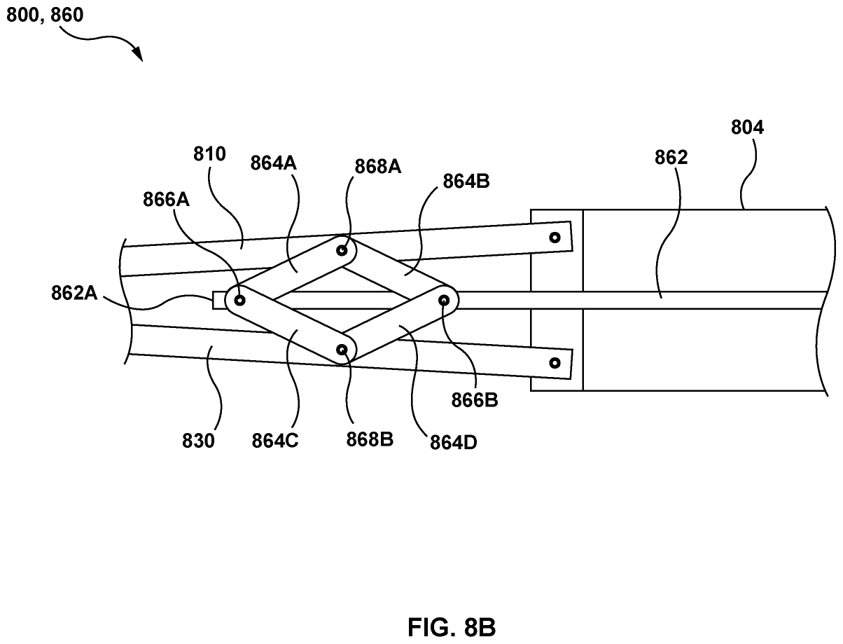

As mentioned previously, the jack mechanism 160 may be any mechanism known to those skilled in the art. The jack mechanism 160 may be actuated by any actuation mechanism known to those skilled in the art. FIGS. 8A-8B illustrate an example embodiment, wherein the steerable cutting device 800 has a jack mechanism 860 that includes a central rod 862 and a plurality of linkages 864. As can be seen in the open configuration in FIG. 8A, the central rod 862 extends distally from the handle (not shown) of the steerable cutting device 800 and extends between the first and second jaws 810, 830 of the steerable cutting device 800. A distal end 862A of the central rod 862 terminates between the first and second jaws 810, 830. The central rod 862 includes threads 865. The distal end 862A of the central rod 862 is coupled to the first and second jaws 810, 830 by a plurality of linkages 864. The linkages 864 are rods that are coupled to the central rod 862 at central rod linkage hubs 866A, 866B and to one of the first jaw 810 or the second jaw 830.

As shown in FIG. 8A, exactly four linkages 864 can be coupled in a substantially diamond-shape to exactly two central rod linkage hubs 866 and exactly two jaw hinge points 868. More specifically, a first linkage 864A is coupled to the central rod 862 at a first central rod linkage hub 866A and the first jaw 810 at a first jaw hinge point 868A, a second linkage 864B is coupled to the central rod 862 at a second central rod linkage hub 866B and the first jaw 810 at the first jaw hinge point 868A, a third linkage 864C is coupled to the central rod 862 at the first central rod linkage hub 866A and the second jaw 830 at a second jaw hinge point 868B, and a fourth linkage 864D is coupled to the central rod 862 at the second central rod linkage hub 866B and the second jaw 830 at the second jaw hinge point 868B. The first central rod linkage hub 866A is disposed distal to the second linkage hub 866B, as shown in FIG. 8A. The first central rod linkage hub 866A is fixedly connected to the central rod 862. The second central rod linkage hub 866B is threaded and threadably engaged with the threaded central rod 862 such that rotation of the threaded central rod 862 causes the second central rod linkage hub 866B to slide linearly along the central rod 862, discussed in further detail below.

FIG. 8B shows the steerable cutting device 800 of FIG. 8A in the closed configuration. To actuate the jack mechanism 860 and transition the steerable cutting device 800 from the open configuration of FIG. 8A to the closed configuration of FIG. 8B, the central rod 862 is rotated such that the threaded second central rod linkage hub 866B begins to slide proximally along the central rod 862 towards the catheter shaft 804 while the first central rod linkage hub 866A remains fixed at the distal end 862A of the central rod 862. As the second linkage hub 866B moves proximally, the second central rod linkage hub 866B moves away from the first central rod linkage hub 866A. The linkages 864A-D rotate at the first and second jaw hinge points 868A, 868B and the first and second central rod linkage hubs 866A, 866B, which in turn, causes the linkages 864A-864D to flatten or come together. This movement causes the first and second jaw hinge points 868A, 868B to move closer together, which in turn, causes the first and second jaws 810, 830 to move closer together until the jaws 810, 830 are in the closed configuration, as shown in FIG. 8B. However, this is not meant to be limiting, as the linkages and hinge points of the jack mechanism can be arranged in any configuration known to those in the art.

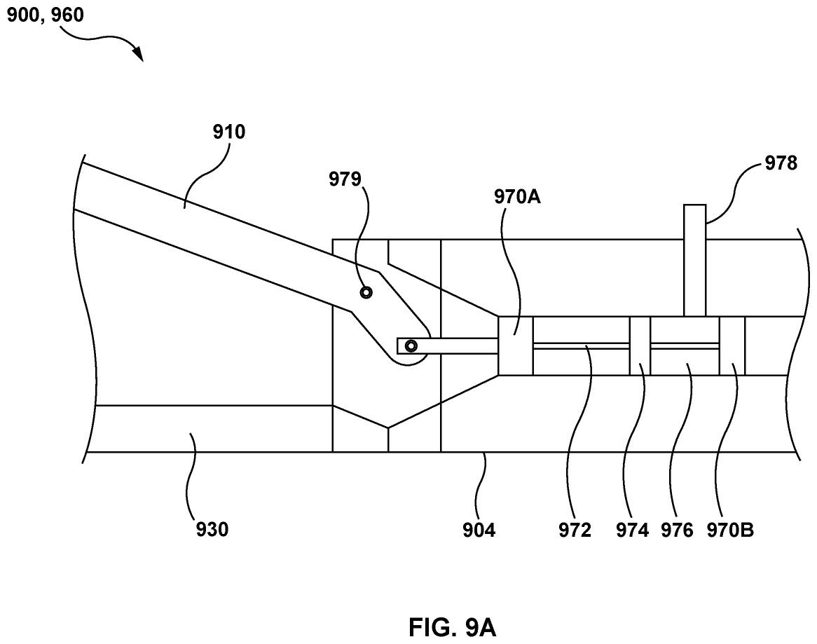

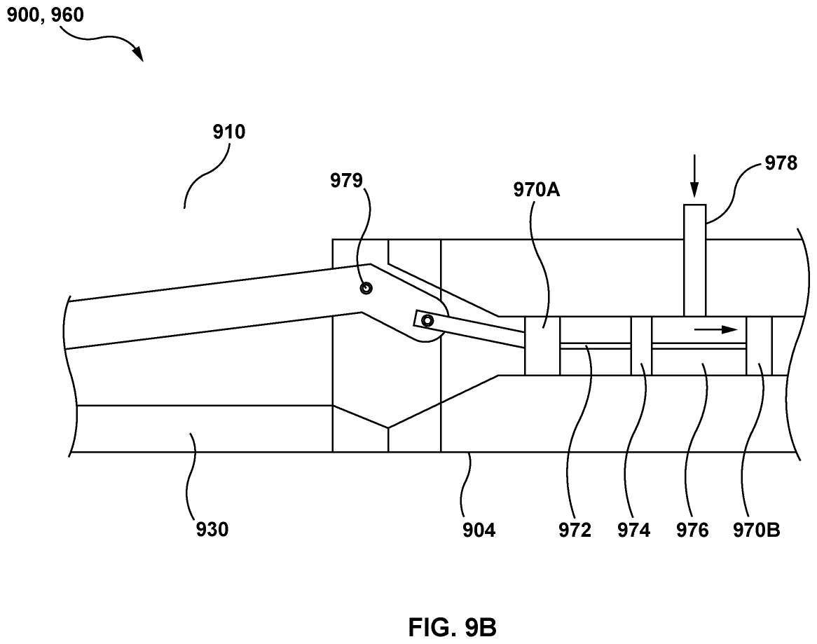

FIGS. 9A-9B show another embodiment, wherein the steerable cutting device 900 includes a hydraulic jack mechanism 960. The hydraulic jack mechanism includes a distal piston 970A, a proximal piston 970B, a cable 972, a seal 974, a hydraulic chamber 976, a hydraulic chamber port 978 and a hinge point 979 on the first jaw 910. The distal piston 970A and the proximal piston 970B are spaced apart from one another within the catheter shaft 904 of the steerable cutting device 900. The seal 974 is disposed in between the distal piston 970A and the proximal piston 970B such that the hydraulic chamber 976 is defined between the seal 974 and the proximal piston 970B, as shown in FIG. 9A. The cable 972 couples the proximal piston 970B to the distal piston 970A. The distal piston 970A is coupled to the first jaw 910 of the steerable cutting device 900. To actuate the hydraulic jack mechanism 960 and transition the steerable cutting device 900 from the open configuration of FIG. 9A to the closed configuration of FIG. 9B, hydraulic pressure is applied into the hydraulic chamber 976 via a hydraulic chamber port 978. Although shown simply extending out of the catheter, the port 978 may open into the hydraulic chamber 976 and be coupled to a hydraulic line (not show) or other means to have hydraulic fluid transported to the hydraulic chamber 976. The pressure causes the proximal piston 970B to slide proximally, which pulls the distal piston 970A proximally via the cable 972. The proximal movement of the distal piston 970A causes the distal end (not shown) of the first jaw 910 to move towards the distal end (not shown) of the second jaw 930 via the hinge point 979 until the jaws 910, 930 are in the closed configuration, as shown in FIG. 9B. The second jaw 930 remains fixed and does not move in response to the actuation of the hydraulic jack mechanism 960.

It should be understood that various embodiments disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single device or component for purposes of clarity, it should be understood that the techniques of this disclosure may be performed by a combination of devices or components.

1. A steerable leaflet resection device comprising:
a first jaw disposed at a distal end of the leaflet resection device;
a second jaw coupled to the first jaw, wherein the first jaw and the second jaw are configured to move relative to each other into a closed configuration wherein distal ends of the first and second jaws are disposed adjacent to each other such as to secure a leaflet of a valve therebetween and an open configuration wherein the distal ends of the first and second jaws spaced apart from each other such as to enable the first and second jaws to move relative to the leaflet with the leaflet disposed therebetween;
a first cutter configured to slide relative to the first and second jaws; and
a second cutter configured to slide relative to the first and second jaws,
wherein the first and second jaws are steerable into a first position wherein the first and second jaws are bent such that sliding the first cutter relative to the first and second jaws cuts the leaflet in a first curved path; and
wherein the first and second jaws are steerable into a second position different than the first position wherein the first and second jaws are bent such that sliding the second cutter relative to the first and second jaws cuts the leaflet in a second curved path different than the first curved path.

2. The steerable leaflet resection device of claim 1, wherein the first cutter is coupled to the first jaw and the second cutter is coupled to the second jaw.

3. The steerable leaflet resection device of claim 1, wherein at least one of the first jaw and the second jaw comprising a hollow, discontinuous tube forming a longitudinal groove facing the other one of the first jaw and the second jaw, wherein at least one of the first cutter and the second cutter is slidably disposed within the tube such that least one of a first blade of the first cutter and a second blade of the second cutter is disposed through the longitudinal groove towards the other one of the first jaw and the second jaw.

4. The steerable leaflet resection device of claim 1, further comprising an anchoring tip disposed between the first jaw and the second jaw, wherein the anchoring tip is configured to secure the leaflet when the first and second jaws are in the closed configuration.

5. The steerable leaflet resection device of claim 4, wherein the anchoring tip extends from a distal end of the first jaw towards a distal end of the second jaw or from the distal end of the second jaw towards the distal end of the first jaw.

6. The steerable resection device of claim 1, further comprising at least a first steering wire and a second steering wire configured to bend the first and second jaws into the first position and the second position.

7. The steerable leaflet resection device of claim 1, further comprising a first steering wire coupled to a first side of the first jaw, a second steering wire coupled to a second side of the first jaw, a third steering wire coupled to a first side of the second jaw, and a fourth steering wire coupled to a second side of the second jaw, wherein tensioning the first and third steering wires bends the first and second jaws into the first position and tensioning the second and fourth steering wire bends the first and second jaws into the second position.

8. The steerable leaflet resection device of claim 1 wherein:
the first jaw comprises a first hollow, discontinuous tube forming a first cavity and a first longitudinal groove facing the second jaw;
the second jaw comprises a second hollow, discontinuous tube forming a second cavity and a second longitudinal groove facing the first jaw;
the first cutter comprises a first base slidably disposed within the first cavity and a first blade extending through the first longitudinal groove towards the second jaw; and
the second cutter comprises a second base slidably disposed within the second cavity and a second blade extending through the second longitudinal groove towards the first jaw.

9. The steerable leaflet resection device of claim 8, wherein the first blade and the second blade comprises sharp tips configured to puncture a leaflet.

10. The steerable leaflet resection device of claim 8, wherein the first blade and the second blade comprise sharpened edges comprised to cut through a leaflet as the first blade or the second blade is moved relative to the leaflet with the leaflet captured between the first and second jaws.

11. A method of resecting a portion of a leaflet of a valve of interest comprising:
tracking a steerable leaflet resection device through a vasculature of a patient to a valve of interest, wherein the steerable leaflet resection device includes a first jaw, a second jaw, a first cutter, and a second cutter;
positioning the steerable leaflet resection device adjacent a free edge of a leaflet of the valve of interest;
advancing the steerable leaflet resection device with the first and second jaws in an open configuration over the leaflet such that the leaflet is disposed between the first and second jaws;
closing the first and second jaws to a closed configuration to secure the leaflet between the first jaw and the second jaw;
bending the first jaw and the second jaw into a first position such that the first jaw and the second jaw are curved into a first curve;
actuating the first cutter such that it moves along a first curved path following the first curve to cut the leaflet along a first cut following the first curved path;
bending the first jaw and the second jaw into a second position different that the first position such that the first jaw and the second jaw are curved into a second curve; and
actuating the second cutter such that it moves along a second curved path following the second curve to cut the leaflet along a second cut following the first second path.

12. The method of claim 11, wherein the steerable leaflet resection device further includes an anchoring tip extending between distal ends of the first and second jaws, wherein closing the first and second jaws to the closed configuration causes the anchoring tip to pinch the leaflet to secure the leaflet between the first and second jaws.

13. The method of claim 11, wherein bending the first jaw and the second jaw into the first position occurs before closing the first jaw and the second jaw to secure the leaflet therebetween.

14. The method of claim 11, wherein bending the first jaw and the second jaw into the first position occurs after closing the first jaw and the second jaw to secure the leaflet therebetween.

15. The method of claim 11, wherein bending the first jaw and the second jaw into the first position comprises pulling steering wires coupled to first sides of the first jaw and the second jaw.

16. The method claim 15, wherein bending the first jaw and the second jaw into the second position comprises pulling steering wires coupled to second sides of the first jaw and the second jaw.

17. The method of claim 11, wherein the valve of interest is a native valve or a previously implanted transcatheter valve prosthesis or a previously implanted surgical valve prosthesis.

18. The method of claim 11, wherein the steerable leaflet resection device is configured to remove at least 50% of the leaflet, or at least 60% of the leaflet, or at least 70% of the leaflet, or at least 80% of the leaflet.

19. The method of claim 11, further comprising retracting the steerable leaflet resection device in the closed position with a resected portion of the leaflet disposed between the first and second jaws in the closed position after making the second cut.

20. The method of claim 11, wherein the steerable leaflet resection device is tracked to the valve of interest with the first and second jaws in the closed configuration.
